(19) United States
(12) Patent Application Publication
(10) Pub. No.: US 2025/0180448 A1
(43) Pub. Date: Jun. 5, 2025

(54) SAMPLE PREPARATION SYSTEM AND SAMPLE PREPARATION METHOD

(71) Applicant: SONY GROUP CORPORATION, TOKYO (JP)

(72) Inventors: YOSHIAKI KATO, TOKYO (JP); TOSHIHARU OKA, TOKYO (JP)

(21) Appl. No.: 18/842,264

(22) PCT Filed: Feb. 22, 2023
(86) PCT No.: PCT/JP2023/006377

§ 371 Date: Aug. 28, 2024

(30) Foreign Application Priority Data
Mar. 7, 2022 (JP) 2022-034401

(51) Int. Cl. G01N 1/40; C12M 1/26; C12M 1/36

(52) U.S. Cl. CPC G01N 1/4005; C12M 33/14; C12M 41/48

(57) ABSTRACT

A main object is to provide a technique of easily performing prewashing in a completely closed sample preparation system. The present technology provides a sample preparation method including: a buffer solution supply step of supplying a buffer solution from a buffer solution container to an empty container; a transfer step of transferring a sample from a sample container to a reservoir; a hollow fiber treatment step of passing the sample in the reservoir through a hollow fiber module; and a prewashing step of transferring the buffer solution in the empty container to the sample container and then transferring the buffer solution to the reservoir, the sampling preparation method performing the steps in a sealed state.

### TECHNICAL FIELD

The present technology relates to a sample preparation system and a sample preparation method, and particularly to a sample preparation system and a sample preparation method used for preparing a sample containing a bio-related particle.

### BACKGROUND ART

Sorting or analysis of bio-related particles is performed in various ways. For example, a method of sorting or analyzing a bio-related particle in a closed space or an open space has been developed. As a representative method for the sorting or analysis, for example, flow cytometry (hereinafter also referred to as FCM) can be mentioned. In order to sort or analyze the bio-related particles, cell staining is performed using a fluorescently labeled antibody. The cell staining makes it possible to analyze and/or sort a specific cell population. For example, by using a FITC-labeled CD3 antibody, T cells can be stained, and the stained T cells can be analyzed and/or sorted by, for example, FCM. In the FCM, all cells are analyzed one by one, so that very high definition analysis and/or purification can be performed.

In bio-related particle analysis in which all cells are analyzed one by one, such as FCM, the analysis time correlates with the number of cells to be analyzed. Therefore, a sample containing a large number of cells to be analyzed but few target cells leads to an unnecessary increase in the time required for the analysis of the bio-related particles. Therefore, for example, by roughly purifying a sample to be subjected to the bio-related particle analysis, a ratio of the target cells can be increased. As a result, the analysis time can be shortened.

Several techniques related to such crude purification have been proposed so far. For example, Patent Document 1 below describes “a cell separation method of removing red blood cells from a cell suspension containing the red blood cells and white blood cells and recovering the white blood cells”. The method is described as a method “including: (A) introducing a cell suspension from an inlet of a cell separation filter obtained by filling a container with a cell separation material, and capturing white blood cells on the cell separation filter; (B) introducing a washing liquid from the inlet of the cell separation filter, and washing the cell separation filter; and (C) introducing a recovery liquid from an outlet of the cell separation filter, and recovering the white blood cells captured on the cell separation filter, in which the washing liquid and the recovery liquid contain a divalent cation chelator, and (a concentration (W/V) of the divalent cation chelator in the washing liquid):(a concentration (W/V) of the divalent cation chelator in the recovery liquid) is 1:200 to 200:1”.

### CITATION LIST

### Patent Document

Patent Document 1: WO 2014/119529 A

### SUMMARY OF THE INVENTION

### Problems to be Solved by the Invention

As described above, in order to increase a content ratio of target particles to be sorted or analyzed, pretreatment for increasing the content ratio of the target particles may be performed before sorting or analysis. Furthermore, pretreatment such as staining of the target particles to be sorted or analyzed may be performed. In the pretreatment of such a sample, a sample remaining in a sample container, a flow path, or the like, that is, a dead volume may be a problem. For example, in a case where rare cells are to be analyzed, as an amount of the sample to be handled is smaller, the problem of the dead volume becomes more remarkable.

In order to reduce the dead volume and improve the accuracy of a sample input amount and a sample preparation efficiency, it is very effective to prewash the sample remaining in the sample container, the flow path, or the like with a buffer solution. However, there is a problem that it is difficult to perform prewashing in a so-called completely closed system in which a substance used for preparation of a sample or the like is configured not to be in fluid communication with an external environment. In particular, in a device that performs a complicated preparation operation of a sample using a hollow fiber, since the number of samples to be handled is relatively small, there is a problem that it is difficult to perform prewashing although it is very important to perform prewashing.

Therefore, a main object of the present technology is to provide a technique of easily performing prewashing in a completely closed sample preparation system.

### Solutions to Problems

In the present technology, first, there is provided a sample preparation method including:
a buffer solution supply step of supplying a buffer solution from a buffer solution container to an empty container;
a transfer step of transferring a sample from a sample container to a reservoir;
a hollow fiber treatment step of passing the sample in the reservoir through a hollow fiber module; and
a prewashing step of transferring the buffer solution in the empty container to the sample container and then transferring the buffer solution to the reservoir,
the sampling preparation method performing the steps in a sealed state.

In the sample preparation method according to the present technology, the hollow fiber treatment step and the prewashing step can be performed in any order or simultaneously.

In the hollow fiber treatment step in the sample preparation method according to the present technology, a concentration treatment of the sample can be performed.

Furthermore, in the hollow fiber treatment step, a solvent exchange treatment of the sample can also be performed.

The sample preparation method according to the present technology may further includes an incubation step of incubating the sample.

The sample preparation method according to the present technology may further includes a concentration adjustment step of adjusting a concentration of the sample.

The sample preparation method according to the present technology may further includes a recovery step of recovering the sample after the hollow fiber treatment into the reservoir.

In the sample preparation method according to the present technology, the sample may contain a bio-related particle.

In this case, the bio-related particle may include a cell.

In the present technology, next, there is provided a sample preparation kit including:
a sample container connection portion connected to a sample container;
a buffer solution container connection portion connected to a buffer solution container;
a reservoir that accommodates a sample being prepared;
a hollow fiber module through which the sample in the reservoir flows; and
an empty container,
in which the sample container connection portion, the buffer solution container connection portion, the reservoir, the hollow fiber module, and the empty container are connected in a sealed manner, and
the empty container is disposed between the sample container connection portion and the reservoir.

In the present technology, further, there is provided a sample preparation system including:
a sample preparation kit; and
a sample preparation device,
the sample preparation kit including:
a sample container connection portion connected to a sample container;
a buffer solution container connection portion connected to a buffer solution container;
a reservoir that accommodates a sample being prepared;
a hollow fiber module through which the sample in the reservoir flows; and
an empty container, the sample container connection portion, the buffer solution container connection portion, the reservoir, the hollow fiber module, and the empty container being connected in a sealed manner, and the empty container being disposed between the sample container connection portion and the reservoir, and
the sample preparation device including:
a first pump that transfers the sample and the buffer solution from the sample container and the buffer solution container to the reservoir; and
a second pump that circulates the sample between the reservoir and the hollow fiber module.

The first pump of the sample preparation system according to the present technology can also transfer the buffer solution from the buffer solution container to the empty container.

The first pump of the sample preparation system according to the present technology can also transfer the buffer solution from the empty container to the sample container.

The sample preparation system according to the present technology may include a control unit that controls the first pump and/or the second pump.

Control by the control unit may include automatic control.

### BRIEF DESCRIPTION OF DRAWINGS

FIG. 1 is a schematic conceptual diagram schematically illustrating a first embodiment of a sample preparation system 1 according to the present technology.

FIG. 2 is a schematic conceptual diagram schematically illustrating a first embodiment of a sample preparation kit 10 according to the present technology.

FIG. 3 is a schematic conceptual diagram schematically illustrating a second embodiment of the sample preparation system 1 according to the present technology.

FIG. 4 is a schematic conceptual diagram schematically illustrating a second embodiment of the sample preparation kit 10 according to the present technology.

FIG. 5 is a schematic conceptual diagram schematically illustrating a third embodiment of the sample preparation system 1 according to the present technology.

FIG. 6 is a flowchart of a first embodiment of a sample preparation method according to the present technology.

FIG. 7 is a flowchart of a second embodiment of the sample preparation method according to the present technology.

### MODE FOR CARRYING OUT THE INVENTION

Hereinafter, preferred modes for implementing the present technology will be described. Note that embodiments hereinafter described are representative embodiments of the present technology, and the scope of the present technology is not limited only to them. Note that the present technology will be described in the following order.
1. Sample preparation system 1
(1) Overall configuration of sample preparation system 1
(2) Sample
(3) Sample preparation kit 10
(3-1) Sample container connection portion 101
(3-2) Buffer solution container connection portion 102
(3-3) Reservoir 103
(3-4) Hollow fiber module 104
(3-5) Empty container 105
(4) Sample preparation device
(4-1) First pump P01
(4-2) Second pump P02
(4-3) Third pump P03
(5) Control unit
(6) Analysis unit 21
(7) Storage unit
(8) Display unit
(9) User interface
2. Sample preparation method
<First embodiment>
(1) Installation of sample preparation kit 10 on sample preparation device
(2) Connection of sample preparation kit 10 to sample container C01 and buffer solution container C02
(3) Priming step S05
(4) Buffer solution supply step S01
(5) Transfer step S02
(6) Hollow fiber treatment step S03
(7) Prewashing step S04
(8) Recovery step S09
<Second embodiment>
(1) First hollow fiber treatment step S03a (concentration treatment)
(2) Second hollow fiber treatment step S03b (solvent exchange treatment)
(3) Dead volume recovery treatment step S06
(4) Incubation step S07
(5) Third hollow fiber treatment step S03c (concentration treatment)
(6) Fourth hollow fiber treatment step S03d (solvent exchange treatment)
(7) Concentration adjustment step S08
(8) Recovery step S09

### 1. Sample Preparation System 1

### (1) Overall Configuration of Sample Preparation System 1

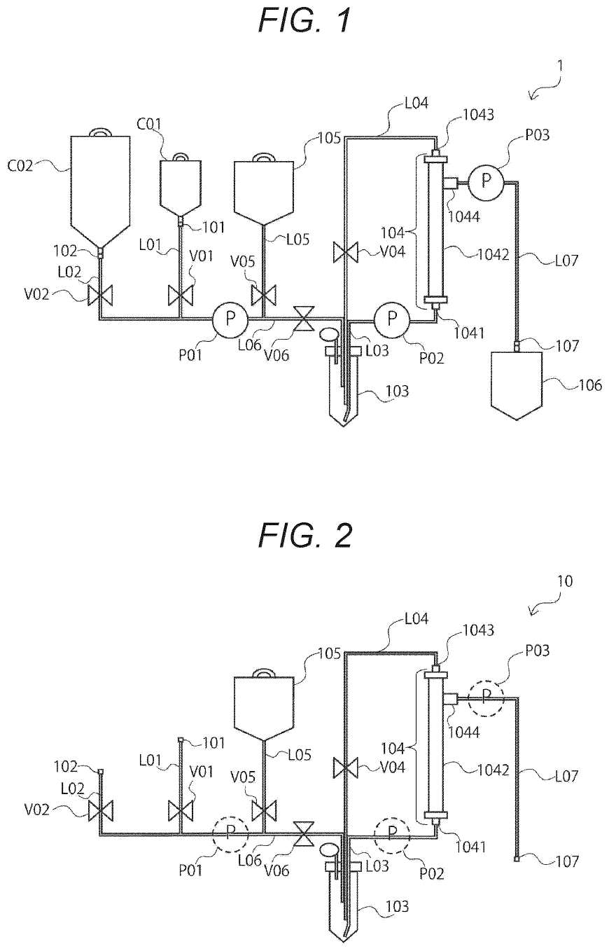

FIG. 1 is a schematic diagram illustrating a first embodiment of a sample preparation system 1 according to the present technology. The sample preparation system 1 according to the present technology includes a sample preparation kit 10 and a sample preparation device. The sample preparation kit 10 includes a sample container connection portion 101, a buffer solution container connection portion 102, a reservoir 103, a hollow fiber module 104, and an empty container 105. Furthermore, the sample preparation device includes a first pump P01 and a second pump P02. In addition, it is also possible to include a control unit, an analysis unit 21, a storage unit, a display unit, a user interface, and the like. Hereinafter, each component and the like will be described in detail.

### (2) Sample

A sample that can be prepared by the sample preparation system 1 according to the present technology is not particularly limited, and examples thereof include a sample to be analyzed or sorted, etc. after preparation, and a reagent for staining the sample.

Examples of the sample include a sample containing particles. The particles may widely include bio-related particles such as cells, microorganisms, and viruses, or synthetic particles such as latex particles, gel particles, and industrial particles.

The bio-related particles include chromosomes constituting various cells, ribosomes, mitochondria, organelles (cell organelles) and the like. The cells include animal cells (for example, hematocyte cells and the like) and plant cells. Examples of the cells include blood cells and tissue cells. Examples of the blood cells include white blood cells (for example, peripheral blood mononuclear cells), red blood cells, and platelets. Examples of the white blood cells include monocytes (macrophages), lymphocytes, neutrophils, basophils, and eosinophils. The cells may be suspension cells such as T cells and B cells. The tissue cells may be, for example, adherent cells separated from adherent cultured cells or tissues. Furthermore, the cells may include tumor cells. The cells may be cultured or uncultured. The bio-related particle may include, for example, a cell mass such as a spheroid and an organoid.

The microorganisms may include bacteria such as Escherichia coli, and fungi such as yeast. The viruses may be, for example, a DNA virus or an RNA virus, and may be a virus with or without an envelope.

The bio-related particles may also include biological macromolecules such as nucleic acids, proteins, and complexes thereof. These biological polymers may be, for example, extracted from the cells or may be contained in blood samples or other liquid samples.

The industrial particles may be, for example, an organic or inorganic polymer material, a metal, or the like. The organic polymer material includes polystyrene, styrene/divinylbenzene, polymethyl methacrylate, and the like. The inorganic polymer material includes glass, silica, a magnetic material, and the like. The metal includes gold colloid, aluminum, and the like. In general, shapes of these particles are normally spherical, but may be non-spherical in the present technology, while a size, mass, and the like thereof are also not particularly limited.

The sample that can be prepared by the sample preparation system 1 according to the present technology is in a liquid state, and examples thereof include a liquid containing the above-described particles. The liquid containing the bio-related particles may be a liquid obtained from a living organism, and examples thereof include a body fluid. Examples of the body fluid include blood, lymph fluid, tissue fluid (for example, intertissue fluid, intercellular fluid, interstitial fluid, and the like), and body cavity fluid (for example, serous cavity fluid, pleural effusion, ascites, pericardial fluid, cerebrospinal fluid (spinal fluid), joint fluid (synovial fluid), and the like). Furthermore, the liquid containing the bio-related particles may be a liquid obtained from these body fluids.

In one embodiment of the present technology, the bio-related particle-containing liquid may be a blood sample, particularly a sample containing white blood cells. The bio-related particle-containing liquid may be, in particular, a blood sample subjected to a red blood cell separation treatment. Note that the blood sample does not have to have red blood cells completely removed, and may include red blood cells. For example, the blood sample may be a blood sample in which an amount of red blood cells in blood collected from a living body is reduced by the separation treatment.

The sample prepared by the sample preparation system 1 according to the present technology can be used as a sample to be subjected to sorting or analysis of bio-related particles. For example, the sample prepared by the sample preparation system 1 of the present technology can be used as a sample to be attached to a device for sorting or analyzing bio-related particles in a closed space or a sample to be attached to a device for sorting or analyzing bio-related particles in an open space. Examples of the device for sorting or analyzing the bio-related particles in the closed space include, but are not limited to, a microparticle sorting device described in Japanese Patent Application Laid-Open No. 2020-76736. Examples of the device for sorting or analyzing the bio-related particles in the open space include, but are not limited to, a microparticle measurement device described in Japanese Patent Application Laid-Open No. 2020-51936. The sample preparation system 1 of the present technology can be used to prepare a sample to be attached to a device that performs such sorting or analysis.

### (3) Sample Preparation Kit 10

FIG. 2 is “a schematic conceptual diagram schematically illustrating a first embodiment of the sample preparation kit 10 according to the present technology”. The sample preparation kit 10 includes the sample container connection portion 101, the buffer solution container connection portion 102, the reservoir 103, the hollow fiber module 104, and the empty container 105. Furthermore, these components are connected in a sealed state via respective flow paths L01 to L08 and the like. The material of each of the flow paths L01 to L08 is not particularly limited, and can be freely selected according to the type and properties of the sample or the like to be used. Furthermore, a form of a dimension and the like of a cross section of each of the flow paths L01 to L08 can be appropriately set according to the type and properties of the sample to be flowed. Specific examples of the flow paths L01 to L08 include tubes.

The sample preparation system 1 of the present technology is a so-called completely closed system configured such that a substance used for preparation of a sample or the like is not in fluid communication with an external environment. The completely closed type can prevent contamination of the sample. For example, in a case where the sample prepared by the sample preparation system 1 of the present technology is a sample containing a bio-related particle, it is very important to prevent contamination.

The sample preparation kit 10 used in the sample preparation system 1 of the present technology is preferably disposable. Contamination of the sample can be prevented by making the kit disposable. Furthermore, by making the kit disposable, it is possible to omit steps such as cleaning in the case of preparing another sample, and it is possible to reduce preparation time and cost.

In the present technology, the sample preparation kit 10 according to the present technology can be distributed. That is, in the sample preparation system 1 according to the present technology, it is possible to make the sample preparation kit 10 disposable by detaching the sample preparation kit 10 through which a substance used for preparation of a sample or the like flows from a sample preparation device as described later and distributing the sample preparation kit.

### (3-1) Sample Container Connection Portion 101

The sample container connection portion 101 is a portion connected to the sample container C01. The sample container C01 is not essential to the sample preparation kit 10 according to the present technology, and a container in which a sample to be prepared is enclosed in advance can be used. For example, in a case where the sample to be prepared is a blood sample, a blood bag or the like in which blood collected from a patient or the like is enclosed after being subjected to a process such as centrifugation as necessary can be used as the sample container C01. That is, as in the first embodiment of the sample preparation kit 10 according to the present technology illustrated in FIG. 2, the sample preparation kit can be distributed in a state where the sample container C01 is not connected to the sample container connection portion 101.

On the other hand, the sample preparation kit 10 according to the present invention may include the sample container C01 in advance. For example, in a case where the sample to be prepared is various drugs, various reagents, or the like (Hereinafter, also referred to as “reagent or the like”.) used for staining or the like, it is also possible to distribute the sample preparation kit 10 with a reagent or the like in a state where the sample container C01 such as a reagent bag is connected to the sample container connection portion 101.

A connection method between the sample container C01 and the sample container connection portion 101 is not particularly limited as long as the sample container and the sample container connection portion can be connected in a sealed state. For example, a lid portion of the sample container C01 includes a puncturable material, and the sample container connection portion 101 includes a flow path such as an injection needle and is configured to be puncturable, so that it is possible to make the connection between the sample container and the sample container connection portion in a sealed state.

The sample container connection portion 101 is connected to the reservoir 103 as described later in a sealed state via the flow path L01 and the flow path L06. A valve V01 can be provided in the flow path L01. Furthermore, the flow path L06 can be provided with a valve V06 and a first pump P01 installation portion that installs the first pump P01 of a sample preparation device as described later. The sample can or cannot flow from the sample container C01 to the reservoir 103 by opening and closing the valves V01 and V06 and driving the first pump P01 as described later.

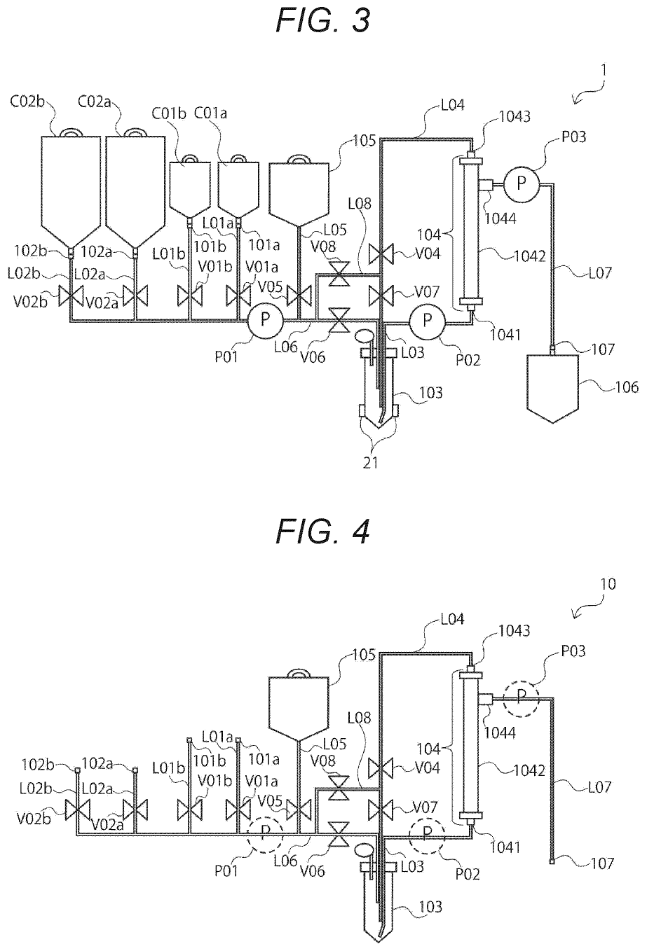

FIG. 3 is a schematic conceptual diagram schematically illustrating a second embodiment of the sample preparation system 1 according to the present technology. In the sample preparation system 1 according to the present technology, as described above, a sample to be analyzed, sorted, or the like after preparation, or a reagent for staining the sample can be used as the sample. Then, these may be used in combination. In this case, a sample container C01a and a reagent container C01b can be used as the sample container C01, and a sample preparation kit 10 according to the present technology can include a sample container connection portion 101a and a reagent container connection portion 101b.

The sample container connection portion 101a is connected to a reservoir 103 as described later in a sealed state via a flow path L01a and a flow path L06. The flow path L01a can be provided with a valve V01a. By opening and closing the valve V01a and a valve V06 and driving a first pump P01 as described later, the flow of the sample from the sample container C01a to the reservoir 103 becomes possible or impossible.

The reagent container connection portion 101b is connected to the reservoir 103 as described later in a sealed state via a flow path L01b and the flow path L06. The flow path L01b can be provided with a valve V01b. By opening and closing the valves V01b and V06 and driving the first pump P01 as described later, the flow of the sample from the reagent container C01b to the reservoir 103 becomes possible or impossible.

Note that, in the second embodiment illustrated in FIGS. 3 and 4, the embodiment in which the two sample container connection portions 101 are provided has been exemplified, but three or more sample container connection portions 101 may be provided. For example, in a case where two or more kinds of reagents are used, for example, in a case where staining or the like on a sample is performed in two stages, it is also possible to provide three sample container connection portions, that is, the sample container connection portion 101a, a first reagent container connection portion 101b, and a second reagent container connection portion (not illustrated).

### (3-2) Buffer Solution Container Connection Portion 102

The buffer solution container connection portion 102 is a portion connected to the buffer solution container C02. The buffer solution container C02 is not essential to the sample preparation kit 10 according to the present technology, and a container in which a buffer solution that can be used for prewashing or sample preparation is enclosed in advance can be prepared and used separately from the sample preparation kit 10. That is, as in the first embodiment of the sample preparation kit 10 according to the present technology illustrated in FIG. 2, the sample preparation kit can be distributed in a state where the buffer solution container C02 is not connected to the buffer solution container connection portion 102.

On the other hand, the sample preparation kit 10 according to the present invention may include the buffer solution container C02 in advance. For example, in a state where the buffer solution container C02 is connected to the buffer solution container connection portion 102, the sample preparation kit 10 with a buffer solution can be distributed.

A connection method between the buffer solution container C02 and the buffer solution container connection portion 102 is not particularly limited as long as the buffer solution container and the buffer solution container connection portion can be connected in a sealed state. For example, a lid portion of the buffer solution container C02 includes a puncturable material, and the buffer solution container connection portion 102 has a flow path such as an injection needle and is configured to be puncturable, so that it is possible to make the connection between the buffer solution container and the buffer solution container connection portion in a sealed state.

The buffer solution container connection portion 102 is connected to the reservoir 103 as described later in a sealed state via a flow path L02 and the flow path L06. A valve V02 can be provided in the flow path L02. The buffer solution can or cannot flow from the buffer solution container C02 to the reservoir 103 by opening and closing the valves V02 and V06 and driving the first pump P01 as described later.

Note that, in the first embodiment illustrated in FIGS. 1 and 2, the embodiment including one buffer solution container connection portion 102 has been exemplified. However, as in the sample preparation systems 1 according to the second embodiment and the third embodiment illustrated in FIGS. 3 to 5, two or more buffer solution container connection portions 102 (102a and 102b) may be provided. For example, in a case where the buffer solution for prewashing the sample container C01a is different from the buffer solution for staining or the like, the buffer solution container connection portions 102 (102a and 102b) can be provided for the required number of buffer solutions.

### (3-3) Reservoir 103

The sample preparation kit 10 according to the present technology includes the reservoir 103 that stores a sample being prepared. The specific form of the reservoir 103 is not particularly limited, and can be freely designed as appropriate according to a state of the sample or the like, such as a cylindrical body, a polygonal cylindrical body having a polygonal cross section (a triangle, a square, or more), a conical body, a polygonal pyramid body having a polygonal cross section (a triangle, a square, or more), or a combination of one or two or more thereof as long as the sample under preparation can be accommodated.

Furthermore, the material constituting the reservoir 103 is not particularly limited, and can be appropriately and freely selected as long as the state of the sample being prepared is not affected. In the present technology, in particular, the reservoir 103 preferably includes resin from the viewpoint of ease of processing and molding. In the present technology, the type or the like of the resin that can be used is not particularly limited, and one or two or more resins that can be applied to accommodate a sample can be appropriately and freely selected and used. Examples thereof include hydrophobic and insulating polymers and copolymers such as polypropylene, polymethyl methacrylate, polystyrene, acrylic, polysulfone, and polytetrafluoroethylene, and blend polymers.

In the present technology, it is particularly preferable to form the reservoir 103 with one or more resins selected from polypropylene, polystyrene, acrylic, and polysulfone among these resins. These resins are suitable for measurement of a blood sample because they have a property of being low coagulation activity for the blood sample.

A flow path for recovering liquid may be connected to the reservoir 103. The liquid recovery flow path can be used, for example, to collect a sample before, during, or after a treatment by the hollow fiber module 104 from the reservoir 103. Furthermore, the liquid recovery flow path can also be used, for example, to collect a sample before, during, or after incubation treatment as described later from the reservoir 103.

The reservoir 103 can be provided with various filters such as a membrane filter and various sensors such as a liquid amount sensor. Furthermore, since the sample can be incubated in the reservoir 103 as described later, for example, a temperature control mechanism for holding a temperature suitable for incubation can be provided.

In the present technology, in a case where a reagent or the like is used, as in the second embodiment illustrated in FIG. 2, the sample container C01a and the reagent, etc. container C01b can be provided as the sample container C01, but a predetermined reagent or the like can be solidified in advance or stored in the reservoir 103 as a liquid. For example, a staining reagent can be placed in the reservoir 103 in advance. Furthermore, for example, a drug for stabilizing a sample or the like can be placed in the reservoir 103 in advance. More specifically, for example, in a case where the sample preparation system 1 according to the present technology is used for preparing a blood sample, for example, blood coagulation can be prevented during sample preparation by placing a drug or the like that inhibits blood coagulation in the reservoir 103 in advance.

In this way, by storing the drug, the reagent, and the like in the reservoir 103 in advance, the reagent, etc. container C01b becomes unnecessary, and it is possible to reduce the size and cost of the system. Furthermore, it is not necessary for the user to replace the reagent or the like, and maintenance of the reagent, etc. container C01b or the like is not necessary, so that the usability can be improved.

### (3-4) Hollow Fiber Module 104

The hollow fiber module 104 includes an inlet 1041, a container 1042, an outlet 1043, and a waste liquid outlet 1044. The container 1042 is filled with a hollow fiber.

The hollow fiber contained in the hollow fiber module 104 can be freely selected according to the type, size, and the like of the sample to be prepared. A material for forming the hollow fiber used in the present technology is not particularly limited. For example, the hollow fiber can be formed using one or two or more kinds of modified polyethersulfone (mPES, mixed cellulose ester (ME), polyethersulfone (PES), polysulfone (PS), and the like.

A pore size expressed as molecular weight cut off (MWCO) of the hollow fiber used in the present technology is also not particularly limited, and can be freely designed according to the type, size, and the like of the sample to be prepared. For example, it can be set to 1 kD or more and 1000 kD or less, preferably 2 kD or more and 900 kD or less, and more preferably 3 kD or more and 800 kD or less. The pore size of the hollow fiber used in the present technology can be, for example, 0.1 μm or more and 1.0 μm or less, preferably 0.15 μm or more and 0.9 μm or less, and more preferably 0.2 μm or more and 0.8 μm or less.

The hollow fiber module 104 is configured to allow the sample in the reservoir 103 to flow. For example, as illustrated in FIG. 1, the hollow fiber module 104 is connected to the reservoir 103 by the flow path L03 and the flow path L04. The flow path L03 and the flow path L04 are also collectively referred to as a circulation flow path.

The sample in the reservoir 103 is supplied to the hollow fiber module 104 through the flow path L03, and then, the sample having passed through the hollow fiber module 104 is returned to the reservoir 103 through the flow path L04. Thus, the sample circulates between the reservoir 103 and the hollow fiber module 104.

As described above, the sample preparation system 1 according to the present technology can preferably include the circulation flow path (the flow path L03 and the flow path L04) for circulating the sample between the reservoir 103 and the hollow fiber module 104.

Note that a direction of the circulation may be opposite. That is, the sample in the reservoir 103 may circulate through the flow path L04 to be supplied to the hollow fiber module 104, and then, the sample having passed through the hollow fiber module 104 may circulate through the flow path L03 to be returned to the reservoir 103.

The flow path L03 can be provided with a second pump installation portion for installing a second pump P02 of the sample preparation device as described later. Furthermore, a valve V04 can be provided in the flow path L04. The sample can be circulated between the reservoir 103 and the hollow fiber module 104 by opening and closing the valve V04 and driving the second pump P02 as described later.

For the hollow fiber module 104, the outlet 1044 through which a waste liquid (also referred to as a permeate liquid in the hollow fiber treatment) generated as a result of the hollow fiber treatment of the sample is discharged and the flow path L07 through which the waste liquid or the permeate liquid flows can also be connected. Furthermore, a permeate liquid recovery container 106 that recovers a waste liquid or a permeate liquid can be provided at a tip of the flow path L07.

The flow path L07 can be provided with a third pump installation portion for installing a third pump P03 of the sample preparation device as described later. The waste liquid or the permeate liquid generated as a result of the hollow fiber treatment of the sample can be recovered into the waste liquid or permeate liquid recovery container 106 by driving the third pump P03 as described later.

The flow path L07 and the waste liquid or permeate liquid recovery container 106 are not essential for the sample preparation kit 10 according to the present technology. For example, in a case where the waste liquid or the permeate liquid is discarded without being used for subsequent analysis or the like, it is not necessary to recover the waste liquid or the permeate liquid from the hollow fiber module 104 in a closed manner. Therefore, the sample preparation kit 10 according to the present technology does not include the flow path L07 and the waste liquid or permeate liquid recovery container 106, and an external flow path or recovery container can be connected as necessary.

On the other hand, in a case where the waste liquid or the permeate liquid is used for subsequent analysis or another application, contamination of the waste liquid or the permeate liquid can be prevented by recovering the waste liquid or the permeate liquid from the hollow fiber module 104 in a closed manner. In such a case, it is preferable that the flow path L07 and the waste liquid or permeate liquid recovery container 106 are provided in a state of being hermetically connected to the sample preparation kit 10 according to the present technology.

Alternatively, the sample preparation kit 10 according to the present technology may not include the waste liquid or permeate liquid recovery container 106, but may include the flow path L07 and a waste liquid or permeate liquid recovery container connection portion 107 for connecting to the waste liquid or permeate liquid recovery container 106. That is, as in the first embodiment of the sample preparation kit 10 according to the present technology illustrated in FIG. 2 and the second embodiment of the sample preparation kit 10 according to the present technology illustrated in FIG. 4, the sample preparation kit can be distributed in a state where the waste liquid or permeate liquid recovery container 106 is not connected to the waste liquid or permeate liquid recovery container connection portion 107. In this case, a sealable container is used as the waste liquid or permeate liquid recovery container 106, a lid portion of the container includes a puncturable material, and the permeate liquid recovery container connection portion 107 has a flow path such as an injection needle and is configured to be puncturable, so that it is possible to make a connection between the container and the permeate liquid recovery container connection portion in a sealed state.

The flow path L04 can be provided with a branch flow path L08 as in the second embodiment illustrated in FIG. 3. At this time, the flow path L04 can include a valve V07 in addition to the valve V04. The branch flow path L08 extends from between the valve V04 and the valve V07 of the flow path L04 to the flow path L06, and is connected to the flow path L06 at a position between the first pump P01 installation portion and the valve V06 on the flow path L06.

The branch flow path L08 branches from the circulation flow path (L03 and L04), and can also be said to lead to the reservoir 103 via the flow path L06. As described above, the sample preparation system 1 according to the present technology can include the branch flow path L08 that branches from the circulation flow path (L03 and L04) and leads to the reservoir 103. The branch flow path L08 can be used for a dead volume recovery treatment as described later.

A valve V08 can be provided on the branch flow path L08. By opening and closing the valve V08, the flow of liquid from the flow path L04 to the flow path L06 or the flow opposite thereto becomes possible or impossible.

### (3-5) Empty Container 105

The sample preparation kit 10 according to the present technology is characterized by including the empty container 105. Then, the empty container 105 is characterized by being disposed between the sample container connection portion 101 and the reservoir 103. Furthermore, the empty container 105 is disposed between the first pump P01 installation portion where the first pump P01 of the sample preparation device as described later is installed and the reservoir 103. The empty container 105 is used for prewashing the sample container C01 and the flow path L01 and the flow path L06 connected to the sample container C01. Details of a prewashing method will be described later.

The empty container 105 is connected to the buffer solution container connection portion 102 in a sealed state via the flow path L05, the flow path L06, and the flow path L02. The buffer solution can or cannot flow from the buffer solution container C02 to the empty container 105 by opening and closing the valves V05 and V02 and driving the first pump P01 as described later.

Furthermore, the empty container 105 is connected to the sample container connection portion 101 in a sealed state via the flow path L05, the flow path L06, and the flow path L01. By opening and closing of the valves V05 and V01 and driving of the first pump P01 as described later, the buffer solution for prewashing can or cannot flow from the empty container 105 to the sample container C01.

Note that, in the first embodiment and the second embodiment illustrated in FIGS. 1 to 4, the embodiments including one empty container 105 has been exemplified. However, as in the sample preparation system 1 according to a third embodiment illustrated in FIG. 5, two or more empty containers 105 (105a and 105b) may be provided. For example, in a case where the buffer solution for prewashing the sample container C01a and the buffer solution for staining are different from each other, the empty containers 105 (105a, 105b) can be provided for the number of necessary buffer solutions for each prewashing.

### (4) Sample Preparation Device

The sample preparation device of the sample preparation system 1 according to the present technology includes the first pump P01 and the second pump P02. Furthermore, the sample preparation device can also include the third pump P03. Hereinafter, each component will be described in detail.

### (4-1) First Pump P01

The first pump P01 is responsible for transferring the sample and the buffer solution from the sample container C01 and the buffer solution container C02 to the reservoir 103. The type of pump that can be used for the first pump P01 is not particularly limited. Examples thereof include a tube pump, and preferably include a peristaltic pump.

The first pump P01 can also be responsible for transferring the buffer solution from the buffer solution container C02 to the empty container 105. Furthermore, the first pump P01 can also transfer the buffer solution from the empty container 105 to the sample container C01. Thus, the sample container C01 and the flow path L01 and the flow path L06 connected to the sample container C01 can be prewashed together.

### (4-2) Second Pump P02

The second pump P02 is responsible for circulation of the sample between the reservoir 103 and the hollow fiber module 104. Thus, the hollow fiber treatment of the sample can be performed.

The type of pump that can be used for the second pump P02 is not particularly limited. Examples thereof include a tube pump, and preferably include a peristaltic pump.

### (4-3) Third Pump P03

The third pump P03 is responsible for transferring the waste liquid or the permeate liquid generated as a result of the hollow fiber treatment of the sample from the hollow fiber module 104 to the waste liquid or permeate liquid recovery container 106. As a result, the waste liquid or the permeate liquid generated as a result of the hollow fiber treatment of the sample can be recovered into the waste liquid or permeate liquid recovery container 106.

The type of pump that can be used for the third pump P03 is not particularly limited. Examples thereof include a tube pump, and preferably include a peristaltic pump.

Note that, as the first pump P01, the second pump P02, and the third pump P03, the same type of pump may be used, or different types of pumps may be used.

### (5) Control Unit

The sample preparation system 1 according to the present technology can include a control unit (not illustrated) that controls the operation of each element constituting the system. The control unit can control, for example, the operation of the pump group and/or the valve group described above. For example, the control unit can automatically control the operation of the pump group and/or the valve group in accordance with a predetermined program.

Furthermore, the control unit can also be configured to receive an analysis result by the analysis unit 21 as described later. The control unit may control the operation of the pump group and/or the valve group according to the analysis result by the analysis unit 21. For example, the control unit can control driving of any one or two or more of the pump groups according to reception of a predetermined analysis result, and specifically, can start or stop driving of any one or two or more of the pump groups.

Furthermore, the control unit can control opening and closing of any one or more of the valve groups according to receiving a predetermined analysis result. As a result, the sample preparation system 1 according to the present technology can control various steps included in a sample preparation method as described later on the basis of an analysis result by the analysis unit 21 as described later, and can control, for example, start or end of the various steps.

Preferably, the sample preparation system 1 according to the present technology can be configured to control a flow operation of a sample to the hollow fiber module 104 on the basis of an analysis result by the analysis unit 21 as described later. More specifically, for example, when the sample in the reservoir has a desired concentration, the flow of the sample to the hollow fiber module 104 as described later can be automatically terminated.

The control unit may be configured as an information processing device (computer), and the function of the control unit can be realized by, for example, a general-purpose computer.

Furthermore, the control unit may be incorporated in the sample preparation device constituting the sample preparation system 1 according to the present technology, but may be connected to the sample preparation kit 10 and the sample preparation device constituting the sample preparation system 1 through a network.

Note that the control unit is not essential to the sample preparation system 1 according to the present technology, and the control can also be performed using an external information processing device or the like.

### (6) Analysis Unit 21

The sample preparation system 1 according to the present technology may include the analysis unit 21. The analysis unit 21 analyzes contents in the reservoir 103. For example, the analysis unit 21 can be configured as a concentration measurement unit that measures the concentration of the sample. The concentration measurement unit can measure the concentration of the sample, for example, by measuring the turbidity of a particle-containing liquid or by measuring light information such as fluorescence generated from the particle-containing liquid. Depending on an analysis result by the analysis unit 21, for example, by continuing or stopping a concentration treatment or the like of the sample using the hollow fiber module 104, the sample in the reservoir 103 can be adjusted to a desired concentration.

Note that the analysis unit 21 is not essential to the sample preparation system 1 according to the present technology, and for example, the contents in the reservoir 103 can be sampled and analyzed using an external analysis device or the like.

### (7) Storage Unit

The sample preparation system 1 according to the present technology can include a storage unit (not illustrated) that stores various data. The storage unit can store, for example, all items related to sample preparation, such as information on the sample analyzed by the analysis unit 21 and records of control processing in the control unit.

The storage unit may be incorporated in the sample preparation device constituting the sample preparation system 1 according to the present technology, or may be connected to the sample preparation kit 10 and the sample preparation device constituting the sample preparation system 1 through a network. Furthermore, the storage unit can be provided in a cloud environment. In this case, each user can share various types of information recorded in the storage unit on the cloud via the network.

Note that, in the sample preparation system 1 according to the present technology, the storage unit is not essential, and various data can be stored using an external storage device or the like.

### (8) Display Unit

The sample preparation system 1 according to the present technology can include a display unit (not illustrated) that displays various types of information. The display unit can display all items related to sample preparation, such as information on the sample analyzed by the analysis unit 21 and contents of control processing in the control unit.

The display unit may be incorporated in the sample preparation device constituting the sample preparation system 1 according to the present technology, or may be connected to the sample preparation kit 10 and the sample preparation device constituting the sample preparation system 1 through a network.

In the present technology, the display unit is not essential, and an external display device may be connected. As the display unit, for example, a display, a printer, or the like can be used.

### (9) User Interface

The sample preparation system 1 according to the present technology can include a user interface (not illustrated) for operation by the user such as an operator. The user can access each component through the user interface to control each component of the sample preparation system 1 according to the present technology. For example, the user interface can set items to be displayed on the display unit, and can set conditions of the hollow fiber treatment.

The user interface may be incorporated in the sample preparation device constituting the sample preparation system 1 according to the present technology, or may be connected to the sample preparation kit 10 and the sample preparation device constituting the sample preparation system 1 through a network.

In the sample preparation system 1 according to the present technology, the user interface is not essential, and an external operation device may be connected. As the user interface, for example, a mouse, a keyboard and the like may be used.

### 2. Sample Preparation Method

The sample preparation method according to the present technology is a method of performing a buffer solution supply step S01, a transfer step S02, a hollow fiber treatment step S03, and a prewashing step S04. Furthermore, a priming step S05, a dead volume recovery treatment step S06, an incubation step S07, a concentration adjustment step S08, a recovery step S09, and the like can be performed as necessary. Hereinafter, each step will be described in detail.

Note that each step can be performed, for example, by controlling a predetermined valve and a predetermined pump as follows. Note that the following control of the valve and the pump can be performed by a user, but can also be automatically performed by the sample preparation system 1 itself (particularly, the control unit) according to the present technology.

### First Embodiment

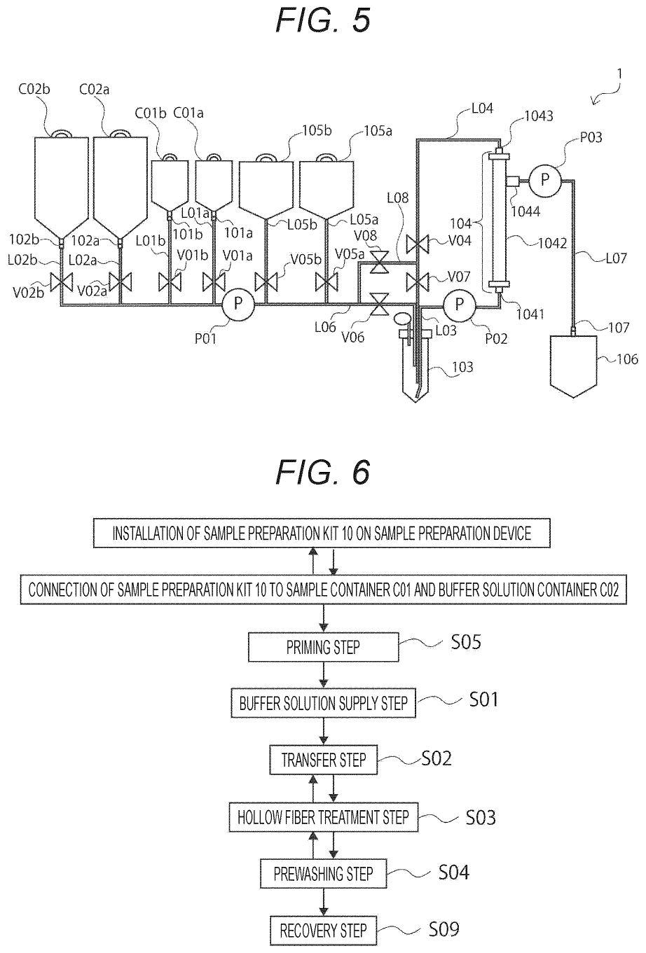

FIG. 6 is a flowchart of a first embodiment of the sample preparation method according to the present technology. The sample preparation method according to the first embodiment is a method of performing the priming step S05, the buffer solution supply step S01, the transfer step S02, the hollow fiber treatment step S03, the prewashing step S04, and the recovery step S09. Hereinafter, each step of the sample preparation method according to the first embodiment will be described with reference to the sample preparation system 1 according to the first embodiment illustrated in FIG. 1.

### (1) Installation of Sample Preparation Kit 10 on Sample Preparation Device

First, the sample preparation kit 10 is installed in the sample preparation device. The sample preparation kit 10 can be distributed by itself, and in particular, in the case of handling a sample containing a bio-related particle as a sample, and the like, it is preferable that the sample preparation kit 10 through which the sample containing the bio-related particle flows is disposable also in order to prevent pollution and contamination.

### (2) Connection of Sample Preparation Kit 10 to Sample Container C01 and Buffer Solution Container C02

After the sample preparation kit 10 is installed in the sample preparation device, the sample container C01 and the buffer solution container C02 are connected to the sample preparation kit 10. For the connection, it is preferable to perform the connection while maintaining the closed state also in order to prevent external contamination. For example, lid portions of the sample container C01 and the buffer solution container C02 include a puncturable material, and the sample container connection portion 101 is configured of an injection needle or the like, so that it is possible to make the connection between the sample container and the buffer solution container, and the sample preparation kit in a sealed state. Note that the “Installation of sample preparation kit 10 on sample preparation device” in the above (1) and the “Connection of sample preparation kit 10 to sample container C01 and buffer solution container C02” in the above (2) can be performed in any order.

### (3) Priming Step S05

The priming step S05 is a step of filling the flow paths L01 to L07 of the sample preparation kit 10 and the hollow fiber module 104 with a buffer solution. By performing the priming step S05, for example, in a case where a sample containing a bio-related particle is used as the sample, it is possible to suppress flow path blockage or the like due to non-specific binding between the bio-related particle and the flow paths L01 to L07 or the like.

In the priming step S05, for example, when the first pump P01 is driven in a state where the valves V02 and V06 are opened, the flow path L02, the flow path L06, and the reservoir 103 are primed with the buffer solution.

Furthermore, when the second pump P02 is driven with the valves V02 and V06 closed and V04 opened, the hollow fiber module 104 is primed with the buffer solution filled in the reservoir 103. In a case where the buffer solution in the reservoir 103 is likely to be depleted, the valves V02 and V06 are opened, and the first pump P01 is driven to compensate for the shortage of the buffer solution.

Moreover, in a case where the third pump P03 is driven in a state where the valves V02 and V06 are opened while the V04 is closed, the flow path L07 is primed with the buffer solution. In a case where the buffer solution in the reservoir 103 is likely to be depleted, the valves V02 and V06 are opened, and the first pump P01 is driven to compensate for the shortage of the buffer solution. Note that, at the end of the priming step, the reservoir 103 is not necessarily filled with the buffer solution as long as the buffer solution is not depleted.

### (4) Buffer Solution Supply Step S01

The buffer solution supply step S01 is a step of supplying a part of the buffer solution from the buffer solution container C02 to the empty container 105. For example, the buffer solution can be supplied from the buffer solution container C02 to the empty container 105 by driving the first pump P01 in a state where the valves V02 and V05 are opened.

### (5) Transfer Step S02

The transfer step S02 is a step of transferring the sample from the sample container C01 to the reservoir 103. For example, the sample can be transferred from the sample container C01 to the reservoir 103 by driving the first pump P01 in a state where the valves V01 and V06 are opened.

The transfer step S02 ends when the sample disappears from the sample container C01.

### (6) Hollow Fiber Treatment Step S03

The hollow fiber treatment step S03 is a step of passing the sample in the reservoir 103 through the hollow fiber module and circulating the sample to perform the hollow fiber treatment. For example, by driving the second pump P02 and the third pump P03 with the valve V04 opened, the sample in the reservoir 103 enters the container 1042 from the inlet 1041 of the hollow fiber module 104 through the flow path L03, and then, the sample passing through the container 1042 exits the outlet 1043 and returns to the reservoir 103 through the flow path L04. Thus, the sample is concentrated by the hollow fiber in the container 1042. The waste liquid generated by the concentration is discharged from the waste liquid outlet 1044, passes through the flow path L07, and is recovered into the waste liquid or permeate liquid recovery container 106.

Note that the hollow fiber treatment step S03 can be started in the middle of the transfer step S02. For example, it is also possible to start the hollow fiber treatment step S03 when an amount of the sample in the reservoir 103 reaches a prescribed amount while performing the transfer step S02, and to simultaneously progress the transfer step S02 and the hollow fiber treatment step S03. At this time, it is preferable to proceed the transfer step S02 and the hollow fiber treatment step S03 while adjusting the amount of the sample in the reservoir 103.

In the hollow fiber treatment step S03, a concentration treatment of the sample can be performed. For example, by performing the concentration treatment of a sample containing a bio-related particle, a proportion of a target particle can be increased. As a result, the analysis time of the bio-related particle can be shortened, and the analysis efficiency can be enhanced.

### (7) Prewashing Step S04

The prewashing step S04 is a step of once transferring the buffer solution in the empty container 105 to the sample container C01 and then transferring the buffer solution in the sample container C01 to the reservoir 103. Thus, the sample container C01 and the flow path L01 can be prewashed together using the buffer solution.

In the prewashing step S04, for example, first, the buffer solution in the empty container 105 is transferred to the sample container C01 in an amount necessary for prewashing by reversely driving the first pump P01 in a state where the valve V05 and the valve V01 are opened. Next, the buffer solution in the sample container C01 is transferred to the reservoir 103 by positively driving the first pump P01 in a state where the valve V01 and the valve V06 are opened. Through these operations, the sample container C01 and the flow path L01 can be prewashed together using the buffer solution.

The prewashing step S04 can be performed before or after the hollow fiber treatment step S03 or can be performed simultaneously with the hollow fiber treatment step S03 as long as the transfer step S02 of transferring the sample from the sample container C01 to the reservoir 103 ends. That is, the hollow fiber treatment step S03 and the prewashing step S04 can be performed in any order or simultaneously.

The prewashing step S04 ends when the buffer solution disappears from the sample container C01.

### (8) Recovery Step S09

The recovery step S09 is a step of recovering the sample after the hollow fiber treatment to the reservoir 103. When the prewashing step S04 ends and the amount of the sample in the reservoir 103 reaches an appropriate amount, the hollow fiber treatment step S03 is also ends, and the preparation of the sample is completed in a state where an appropriate amount of the prepared sample is recovered in the reservoir 103.

In the recovery step S09, it is also possible to recover the hollow fiber and the sample remaining in each flow path by performing the dead volume recovery treatment step S06 as described later. Details of the dead volume recovery treatment step S06 will be described later.

### Second Embodiment

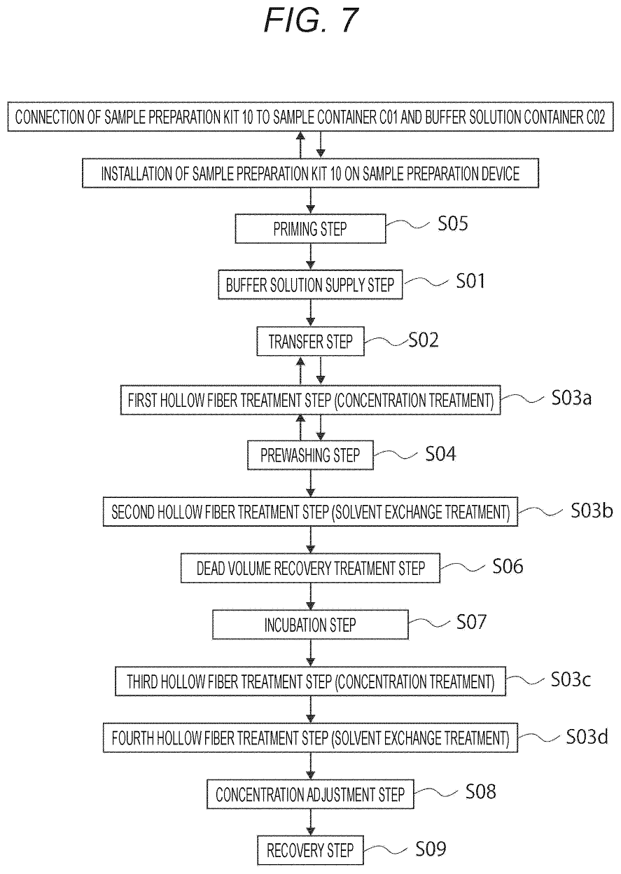

FIG. 7 is a flowchart of a second embodiment of the sample preparation method according to the present technology. The sample preparation method according to the second embodiment is a method of performing a priming step S05, a buffer solution supply step S01, a transfer step S02, a first hollow fiber treatment step S03a, a prewashing step S04, a second hollow fiber treatment step S03b, a dead volume recovery treatment step S06, an incubation step S07, a third hollow fiber treatment step S03c, a fourth hollow fiber treatment step S03d, a concentration adjustment step S08, and a recovery step S09. Hereinafter, each step of the sample preparation method according to the first embodiment will be described with reference to the sample preparation system 1 according to the second embodiment illustrated in FIG. 3.

Note that the preparation step (installation of the sample preparation kit 10 in the sample preparation device, and connection of the sample preparation kit 10 to the sample container C01 and the buffer solution container C02), the priming step S05, the transfer step S02, and the prewashing step S04 are the same as the sample preparation method according to the first embodiment described above, and thus the description thereof is omitted here.

### (1) First Hollow Fiber Treatment Step S03a (Concentration Treatment)

In the first hollow fiber treatment step S03a, the sample is subjected to the hollow fiber treatment to concentrate the sample. Specifically, for example, the sample can be circulated between the reservoir 103 and the hollow fiber module 104 by driving the second pump P02 with the valves V04 and V07 opened. Thus, the sample can be concentrated.

Note that, since other details of the first hollow fiber treatment step S03a are the same as those of the hollow fiber treatment step S03 of the sample preparation method according to the first embodiment, the description thereof will be omitted here.

### (2) Second Hollow Fiber Treatment Step S03b (Solvent Exchange Treatment)

In the sample preparation method according to the present technology, a labeling treatment such as staining of a sample can be performed. Examples of the labeling treatment include a method in which a labeling substance such as a fluorescently labeled antibody is bound to a target particle in a sample, and a method in which a nucleic acid to which a labeling substance is bound is hybridized to a target nucleic acid in a sample.

At this time, it is preferable to exchange the solvent of the sample with a solvent suitable for the labeling treatment. Therefore, a solvent exchange treatment can be performed by performing the second hollow fiber treatment step S03b and performing diafiltration using the hollow fiber module 104.

Specifically, for example, by driving the first pump P01 in a state where the valves V02b and V06 are opened, a labeling treatment buffer solution can be transferred from the buffer solution container C02b into the reservoir 103. As a result, a proportion of the labeling treatment buffer solution in the sample can be increased.

Next, the second pump P02 and the third pump P03 are driven in a state where the valves V04 and V07 are opened simultaneously in parallel with the driving of the first pump P01 in a state where the valves V02b and V06 are opened. By driving the second pump P02 and the third pump P03, the sample in the reservoir 103 enters the container 1042 from the inlet 1041 of the hollow fiber module 104 through the flow path L03, and then, the sample that has passed through the container 1042 exits the outlet 1043 and returns to the reservoir 103 through the flow path L04. As a result, the solvent of the sample is exchanged for the labeling treatment buffer solution. The waste liquid generated by the solvent exchange exits from the waste liquid outlet 1044, passes through the flow path L07, and is recovered into the waste liquid or permeate liquid recovery container 106.

Note that, although the driving of the first pump P01 in a state where the valves V02b and V06 are opened and the driving of the second pump P02 and the third pump P03 in a state where the valves V04 and V07 are opened are simultaneously performed in parallel, these two driving operations may be sequentially performed or may be sequentially repeated.

### (3) Dead Volume Recovery Treatment Step S06

The hollow fiber treatment has a so-called dead volume problem as follows. That is, in the hollow fiber treatment, a treatment such as concentration or diafiltration is performed while circulating the liquid containing the hollow fiber column. Therefore, when the treatment is completed, the sample remains in the flow path other than the reservoir 103 or the hollow fiber module, and an amount of the remaining sample is a dead volume. For example, when the second hollow fiber treatment step S03b (solvent exchange treatment) is completed, a sample remains in the flow path L03, the hollow fiber module 104, and the flow path L04, and the amount of the remaining sample is a dead volume.

The dead volume problem does not become apparent in a case where a large amount of a target substance is recovered in the reservoir 103 by the hollow fiber treatment. However, in bio-related particle analysis such as flow cytometry, an amount of the sample to be used is often relatively small, for example, about 5 mL. This amount is smaller than a recovery amount assumed in a general hollow fiber treatment, and is, for example, about one order of magnitude smaller. Therefore, in the bio-related particle analysis, it is desirable to minimize the dead volume in the hollow fiber treatment.

Furthermore, in the bio-related particle analysis, rare cells such as certain specific immune cells may be analyzed. Therefore, in order to secure the number of cells, it is desirable to minimize the dead volume in the hollow fiber treatment.

Therefore, the problem of the dead volume can be solved by providing the branch flow path L08 that branches from the circulation flow path for circulating the sample between the reservoir 103 and the hollow fiber module 104 and leads to the reservoir 103.

Specifically, first, the first pump P01 is driven in a state where the valves V02b, V07, and V08 are opened. At the time of the driving, the other valves may be closed, or the other pumps may not be driven. As a result, in the flow path L04, the sample from a connection point between the branch flow path L08 and the flow path L04 (a connection point located between the valves V04 and V07) to an end in the reservoir 103 is recovered in the reservoir 103.

Next, the second pump P02 is reversely driven in a state where the pump P1 is opened and the valves V02b, V04, and V08 are opened. Thus, in the flow path L04, the sample in a portion from a connection point between the branch flow path L08 and the flow path L04 (a connection point located between the valves 04 and V07) to the outlet 1043 of the hollow fiber module 104, the sample in the hollow fiber module 104, and the sample in the flow path L03 are recovered in the reservoir 103.

Note that, in the reverse drive of the second pump P02, the third pump P03 can also be reversely driven. This can promote separation of particles contained in the sample from the hollow fiber. The reverse drive of the third pump P03 can be performed at a low speed, for example. Furthermore, the reverse drive of the third pump P03 can be performed, for example, for a short time (e.g. 0.1 s to 5 s, in particular 0.5 s to 3 s). For example, the short-time driving can be performed once or more, for example, 1 to 10 times, preferably 2 to 5 times. By reversely driving the third pump P03 in this manner, the particles are easily separated from the hollow fiber, and the number of particles recovered to the reservoir 103 can be increased.

### (4) Incubation Step S07

As described above, in the sample preparation method according to the present technology, the labeling treatment such as staining of a sample can be performed. For the labeling treatment, the reagent in the reagent container C01b can be introduced into the reservoir 103 and incubated. The incubation conditions (for example, time and temperature) are not particularly limited, and can be appropriately set according to the type of sample or reagent, and the like.

Specifically, for example, by driving the pump P1 in a state where the valves V01b and V06 are opened, the reagent in the sample container C01b is supplied into the reservoir 103 through the flow paths L01b and L06, and incubation is performed.

### (5) Third Hollow Fiber Treatment Step S03c (Concentration Treatment)

In the third hollow fiber treatment step S03c, the solvent-exchanged sample is subjected to the hollow fiber treatment to concentrate the solvent-exchanged sample. Specifically, for example, the sample can be circulated between the reservoir 103 and the hollow fiber module 104 by driving the second pump P02 with the valves V04 and V07 opened. This makes it possible to concentrate the solvent-exchanged sample.

Note that, since other details of the third hollow fiber treatment step S03c are the same as those of the hollow fiber treatment step S03 of the sample preparation method according to the first embodiment, the description thereof will be omitted here.

### (6) Fourth Hollow Fiber Treatment Step S03d (Solvent Exchange Treatment)

In the fourth hollow fiber treatment step S03d, a solvent exchange treatment can be performed to return the solvent of the sample exchanged for the solvent for the labeling treatment to the original solvent.

Specifically, for example, the buffer solution can be transferred from a buffer solution container C02a into the reservoir 103 by driving the first pump P01 in a state where the valves V02a and V06 are opened. As a result, a proportion of the labeling treatment buffer solution in the sample is reduced, and a proportion of the original buffer solution can be increased.

Next, the second pump P02 and the third pump P03 are driven in a state where the valves V04 and V07 are opened simultaneously in parallel with the driving of the first pump P01 in a state where the valves V02a and V06 are opened. By driving the second pump P02 and the third pump P03, the sample in the reservoir 103 enters the container 1042 from the inlet 1041 of the hollow fiber module 104 through the flow path L03, and then, the sample that has passed through the container 1042 exits the outlet 1043 and returns to the reservoir 103 through the flow path L04. The solvent of the sample is exchanged for the original buffer solution. The waste liquid generated by the solvent exchange exits from the waste liquid outlet 1044, passes through the flow path L07, and is recovered into the waste liquid or permeate liquid recovery container 106.

Note that, although the driving of the first pump P01 in a state where the valves V02a and V06 are opened and the driving of the second pump P02 and the third pump P03 in a state where the valves V04 and V07 are opened are simultaneously performed in parallel, these two driving operations may be sequentially performed or may be sequentially repeated.

Note that, after the fourth hollow fiber treatment step S03d (solvent exchange treatment), the dead volume recovery treatment step S06 described above can be performed as necessary.

### (7) Concentration Adjustment Step S08

In the sample preparation method according to the present technology, the concentration adjustment step S08 can be performed. The adjustment of the concentration may be, for example, a dilution treatment for decreasing the concentration or a concentration treatment for increasing the concentration.

For example, in order to perform the dilution treatment, the buffer solution can be transferred from the buffer solution container C02a into the reservoir 103 by driving the first pump P01 in a state where the valves V02a and V06 are opened. As a result, the concentration of the sample can be reduced.

Note that, in the dilution treatment, the hollow fiber treatment can be performed by driving the second pump P02 with the valves V04 and V07 opened. As a result, the particles in the sample can be uniformly dispersed. Furthermore, the driving can also prevent particles from being clogged in holes of the hollow fiber.

Furthermore, for example, it is possible to perform a concentration treatment of the sample and increase its concentration by performing the hollow fiber treatment. Since the method of the hollow treatment is the same as the third hollow fiber treatment step S03c (concentration treatment) described above, the description thereof is herein omitted.

### (8) Recovery Step S09

The recovery step S09 is a step of recovering the sample after the hollow fiber treatment to the reservoir 103. When an amount of the sample in the reservoir 103 reaches an appropriate amount, the concentration adjustment step S08 ends, and the sample preparation is completed in a state where an appropriate amount of the prepared sample is recovered in the reservoir 103.

In the recovery step S09, the hollow fiber and the sample remaining in each flow path can be also recovered by performing the dead volume recovery treatment step S06.

Note that the present technology may also have following configurations.
[1]

A sample preparation method including:
a buffer solution supply step of supplying a buffer solution from a buffer solution container to an empty container;
a transfer step of transferring a sample from a sample container to a reservoir;
a hollow fiber treatment step of passing the sample in the reservoir through a hollow fiber module; and
a prewashing step of transferring the buffer solution in the empty container to the sample container and then transferring the buffer solution to the reservoir,
the sampling preparation method performing the steps in a sealed state.
[2]

The sample preparation method according to [1], in which the hollow fiber treatment step and the prewashing step are performed in any order or simultaneously.
[3]

The sample preparation method according to [1] or [2], in which in the hollow fiber treatment step, a concentration treatment of the sample is performed.
[4]

The sample preparation method according to any one of [1] to [3], in which in the hollow fiber treatment step, a solvent exchange treatment of the sample is performed.
[5]

The sample preparation method according to any one of [1] to [4], further including an incubation step of incubating the sample.
[6]

The sample preparation method according to any one of [1] to [5], further including a concentration adjustment step of adjusting a concentration of the sample.
[7]

The sample preparation method according to any one of [1] to [6], further including a recovery step of recovering the sample after the hollow fiber treatment into the reservoir.
[8]

The sample preparation method according to any one of [1] to [7], in which the sample contains a bio-related particle.
[9]

The sample preparation method according to [8], in which the bio-related particle includes a cell.
[10]

A sample preparation kit including:
a sample container connection portion connected to a sample container;
a buffer solution container connection portion connected to a buffer solution container;
a reservoir that accommodates a sample being prepared;
a hollow fiber module through which the sample in the reservoir flows; and
an empty container,
in which the sample container connection portion, the buffer solution container connection portion, the reservoir, the hollow fiber module, and the empty container are connected in a sealed manner, and
the empty container is disposed between the sample container connection portion and the reservoir.
[11]

A sample preparation system including:
a sample preparation kit; and
a sample preparation device,
the sample preparation kit including:
a sample container connection portion connected to a sample container;
a buffer solution container connection portion connected to a buffer solution container;
a reservoir that accommodates a sample being prepared;
a hollow fiber module through which the sample in the reservoir flows; and
an empty container, the sample container connection portion, the buffer solution container connection portion, the reservoir, the hollow fiber module, and the empty container being connected in a sealed manner, and the empty container being disposed between the sample container connection portion and the reservoir, and
the sample preparation device including:
a first pump that transfers the sample and the buffer solution from the sample container and the buffer solution container to the reservoir; and
a second pump that circulates the sample between the reservoir and the hollow fiber module.
[12]

The sample preparation system according to [11], in which the first pump transfers the buffer solution from the buffer solution container to the empty container.
[13]

The sample preparation system according to or [12], in which the first pump transfers the buffer solution from the empty container to the sample container.
[14]

The sample preparation system according to any one of to [13], further including a control unit that controls the first pump and/or the second pump.
[15]

The sample preparation system according to [14], in which control by the control unit includes automatic control.

### REFERENCE SIGNS LIST

1 Sample preparation system
10 Sample preparation kit
101 Sample container connection portion
102 Buffer solution container connection portion
103 Reservoir
104 Hollow fiber module
105 Empty container
P01 First pump
P02 Second pump
P03 Third pump
21 Analysis unit
S05 Priming step
S01 Buffer solution supply step
S02 Transfer step
S03 Hollow fiber treatment step
S04 Prewashing step
S09 Recovery step
S03a First hollow fiber treatment step (concentration treatment)
S03b Second hollow fiber treatment step (solvent exchange treatment)
S06 Dead volume recovery treatment step
S07 Incubation step
S03c Third hollow fiber treatment step (concentration treatment)
S03d Fourth hollow fiber treatment step (solvent exchange treatment)
S08 Concentration adjustment step

1. A sample preparation method comprising:
a buffer solution supply step of supplying a buffer solution from a buffer solution container to an empty container;
a transfer step of transferring a sample from a sample container to a reservoir;
a hollow fiber treatment step of passing the sample in the reservoir through a hollow fiber module; and
a prewashing step of transferring the buffer solution in the empty container to the sample container and then transferring the buffer solution to the reservoir,
the sampling preparation method performing the steps in a sealed state.

2. The sample preparation method according to claim 1, wherein the hollow fiber treatment step and the prewashing step are performed in any order or simultaneously.

3. The sample preparation method according to claim 1, wherein in the hollow fiber treatment step, a concentration treatment of the sample is performed.

4. The sample preparation method according to claim 1, wherein in the hollow fiber treatment step, a solvent exchange treatment of the sample is performed.

5. The sample preparation method according to claim 1, further comprising an incubation step of incubating the sample.

6. The sample preparation method according to claim 1, further comprising a concentration adjustment step of adjusting a concentration of the sample.

7. The sample preparation method according to claim 1, further comprising a recovery step of recovering the sample after the hollow fiber treatment into the reservoir.

8. The sample preparation method according to claim 1, wherein the sample contains a bio-related particle.

9. The sample preparation method according to claim 8, wherein the bio-related particle includes a cell.

10. A sample preparation kit comprising:
a sample container connection portion connected to a sample container;
a buffer solution container connection portion connected to a buffer solution container;
a reservoir that accommodates a sample being prepared;
a hollow fiber module through which the sample in the reservoir flows; and
an empty container,
wherein the sample container connection portion, the buffer solution container connection portion, the reservoir, the hollow fiber module, and the empty container are connected in a sealed manner, and
the empty container is disposed between the sample container connection portion and the reservoir.

11. A sample preparation system comprising:
a sample preparation kit; and
a sample preparation device,
the sample preparation kit including:
a sample container connection portion connected to a sample container;
a buffer solution container connection portion connected to a buffer solution container;
a reservoir that accommodates a sample being prepared;
a hollow fiber module through which the sample in the reservoir flows; and
an empty container, the sample container connection portion, the buffer solution container connection portion, the reservoir, the hollow fiber module, and the empty container being connected in a sealed manner, and the empty container being disposed between the sample container connection portion and the reservoir, and
the sample preparation device including:
a first pump that transfers the sample and the buffer solution from the sample container and the buffer solution container to the reservoir; and
a second pump that circulates the sample between the reservoir and the hollow fiber module.

12. The sample preparation system of claim 11, wherein the first pump transfers the buffer solution from the buffer solution container to the empty container.

13. The sample preparation system of claim 11, wherein the first pump transfers the buffer solution from the empty container to the sample container.

14. The sample preparation system according to claim 11, further comprising a control unit that controls the first pump and/or the second pump.

15. The sample preparation system according to claim 14, wherein control by the control unit includes automatic control.
